(19) United States
(12) Patent Application Publication
(10) Pub. No.: US 2024/0138946 A1
(43) Pub. Date: May 2, 2024

(54) STEERABLE SHEATH WITH ROBOTIC HANDLE STAND

(71) Applicant: Nextern Innovation, LLC, White Bear Lake, MN (US)

(72) Inventors: John Swoyer, Blaine, MN (US); Steve Gigl, Crystal, MN (US)

(73) Assignee: Nextern Innovation, LLC, White Bear Lake, MN (US)

(21) Appl. No.: 18/547,848

(22) PCT Filed: Feb. 25, 2022
(86) PCT No.: PCT/US22/70846

§ 371 Date: Aug. 24, 2023

Related U.S. Application Data
(63) Continuation of application No. 17/453,988, filed on Nov. 8, 2021.
Application No. PCT/US22/70846 is a continuation of application No. 17/305,856, filed on Jul. 15, 2021, now abandoned.
(60) Provisional application No. 63/111,408, filed on Nov. 9, 2020.
(60) Provisional application No. 63/154,192, filed on Feb. 26, 2021.
(60) Provisional application No. 63/053,197, filed on Jul. 17, 2020.
(60) Provisional application No. 63/154,192, filed on Feb. 26, 2021.

(51) Int. Cl. A61B 34/00; A61B 34/30; A61M 25/01; A61M 25/09

(52) U.S. Cl. CPC A61B 34/71; A61B 34/30; A61B 34/76; A61M 25/0116; A61M 25/0147; A61M 25/09; A61B 2034/301; A61B 2034/715; A61M 2205/3553

(57) ABSTRACT

Apparatus and associated methods relate to a robotic catheter cradle having a receiving module configured to releasably receive a steerable sheath after a distal end of the steerable sheath has been manually introduced into a patient independent of the receiving module. In an illustrative example, a steering drive module may be operably coupled to the robotic catheter cradle to robotically operate at least one interface of the handle to selectively actuate at least one guidewire of the steerable sheath such that a distal end of the steerable sheath is controllably deflected. An axial drive module may, for example, be operably coupled to the robotic catheter cradle to robotically translate the steerable sheath. Various embodiments may advantageously enable a human operator to remove the steerable sheath into a manual mode that permits operation mechanically independent of the robotic catheter cradle without withdrawing the distal end from the patient.

### CROSS-REFERENCE TO RELATED APPLICATIONS

This application claims the benefit of U.S. Provisional Application Ser. No. 63/154,192, titled “Steerable Sheath with Robotic Handle Stand,” filed by John Swoyer, et al., on Feb. 26, 2021.

This application claims the right of priority to U.S. application Ser. No. 17/453,988, titled “Steerable Tip Catheter with Automatic Tension Apparatus,” filed by John Pocrnich, et al., on Nov. 8, 2021, which application claims the benefit of U.S. Provisional Application Ser. No. 63/111,408, titled “Steerable Tip Catheter with Automatic Tension Apparatus,” filed by John Pocrnich, et al., on Nov. 8, 2020.

This application claims the right of priority to U.S. application Ser. No. 17/305,856, titled “Systems and Methods for Minimally Invasive Delivery and in Vivo Creation of Biomaterial Structures,” filed by John Swoyer, et al., on Jul. 15, 2021; which claims the benefit of U.S. Provisional Application Ser. No. 63/053,197, titled “Systems and Methods for Minimally Invasive Delivery and in Vivo Creation of Biomaterial Structures,” filed by John Swoyer, et al., on Jul. 17, 2020.

This application incorporates the entire contents of the foregoing application(s) herein by reference.

The subject matter of this application may have common inventorship with and/or may be related to the subject matter of U.S. application Ser. No. 16/861,633, titled “Robotically Augmented Catheter Manipulation Handle,” filed by Ryan Douglas, et al., on Apr. 29, 2020, which is a continuation of U.S. application Ser. No. 15/425,982, titled “Robotically Augmented Catheter Manipulation Handle,” filed by Ryan Douglas, et al., on Feb. 6, 2017 and issued as U.S. Pat. No. 10,675,442 on Jun, 9, 2020. U.S.

This application incorporates the entire contents of the foregoing application(s) herein by reference.

### TECHNICAL FIELD

Various embodiments relate generally to steerable medical instruments.

### BACKGROUND

Medical teams have available a wide variety of catheters, to enable provision of the right products for their patients' unique medical needs. For decades, with the help of catheters, medical teams have been able to drain fluids from body cavities, administer medications intravenously, perform surgical procedures, and administer anesthetics, for example. As technology progressed, medical instrument designers provided modern medicine teams with guiding catheters and sheaths. Guiding catheters and sheaths are frequently used in many medical procedures due to their minimally invasive nature. For example, patients undergoing cardiac or other vascular procedures with guiding catheters and sheaths receive a minimally sized surgically placed lumen (opening) to the skin.

Guiding catheters and sheaths, otherwise named “steerable” catheters and sheaths, may employ control wires that pass from the catheter interface through the catheter shaft and terminate at the catheter shaft tip. Tension applied to any of the control wires causes the catheter tip to deflect, giving control of orientation to the catheter tip, for example giving orientation control of the imaging angle of a tip mounted ultrasound transducer. This technology has made more advanced procedures possible using catheter-mounted instruments, benefiting patients with minimally invasive procedures, by entering a patient's body percutaneously or via natural orifices. Further descriptions that reference guided catheters may also apply to guided sheaths.

### SUMMARY

Apparatus and associated methods relate to a robotic catheter cradle having a receiving module configured to releasably receive a steerable sheath after a distal end of the steerable sheath has been manually introduced into a patient independent of the receiving module. In an illustrative example, a steering drive module may be operably coupled to the robotic catheter cradle to robotically operate at least one interface of the handle to selectively actuate at least one guidewire of the steerable sheath such that a distal end of the steerable sheath is controllably deflected. An axial drive module may, for example, be operably coupled to the robotic catheter cradle to robotically translate the steerable sheath. Various embodiments may advantageously enable a human operator to remove the steerable sheath into a manual mode that permits operation mechanically independent of the robotic catheter cradle without withdrawing the distal end from the patient.

Various embodiments may achieve one or more advantages. For example, some embodiments may advantageously be configured for an operator to selectively operate a steerable sheath between a robotic mode and a manual mode mechanically independent of the robotic catheter cradle. Mechanical independence of the steerable sheath from the robotic catheter cradle may, for example, advantageously increase patient safety. Embodiments which allow the operator to rapidly operate the steerable sheath into the manual mode (e.g., without disengaging the conduit from the handle and/or withdrawing the conduit from the patient) may advantageously enable the operator to prevent or reduce damage to the patient from a malfunction of a robotic catheter cradle. Mechanical independence of the steerable sheath from the robotic catheter cradle may, for example, advantageously increase mechanical flexibility. Embodiments which allow the operator to freely operate the steerable sheath between a robotic mode using the robotic catheter cradle and a manual mode (mechanically) independent of the robotic catheter cradle may advantageously achieve an operator-desired mixture of advantages of robotic and manual manipulation.

For example, in some embodiments, a robotic catheter cradle may be mechanically decoupled from (e.g., independent of) an introducer and/or dilator. Such embodiments may, for example, advantageously allow the operator to gain full manual control of the handle and the conduit. For example, various embodiments may advantageously avoid a portion of the conduit from being ‘trapped’ by a robotic introducer and/or aperture through a robotic system.

Some embodiments may, by way of example and not limitation, allow an operator to manually use a steerable sheath (e.g., by a handle and (operating) interface(s)) in a method that is familiar to get a tip of a conduit close to a final target position. The operator may then, for example, advantageously place the handle in the robotic catheter cradle. Accordingly, the robotic catheter cradle may advantageously free the operator such as, by way of example and not limitation, to manipulate other devices, to fine tune final position, to change position of the sheath, or some combination thereof. Various embodiments may, for example, advantageously allow an operator to robotically adjust the catheter during fluoroscopy while avoiding a radiation beam, thereby advantageously minimizing radiation exposure of the operator.

An RCC system may be advantageously configured for steerable ablation, intracardiac echo (ICE), and/or mapping catheter(s). Various embodiments may be advantageously applied and/or configured for use in procedures such as, by way of example and not limitation, structural heart applications. Various embodiments may, for example, advantageously be applied and/or configured for use in pulmonary (e.g., lung) procedures. Various embodiments may, for example, advantageously be applied and/or configured for use in gastrointestinal procedures.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary robotic catheter cradle (RCC) employed in an illustrative use-case scenario in which a human operator selectively operates a steerable sheath between a robotically operated mode and a manual mode that permits operation mechanically independent of the RCC.

FIG. 2 depicts an exemplary RCC and steerable sheath handle.

FIG. 3 depicts an exemplary block diagram of an exemplary RCC and steerable sheath handle.

FIG. 4 is a flowchart illustrating an exemplary method of introducing and operating a steerable sheath in a robotic mode and a manual mode mechanically independent of an RCC.

FIG. 5 is a flowchart illustrating an exemplary method of automatic control of an RCC in a robotic catheter control mode and a mechanically independent manual mode.

Like reference symbols in the various drawings indicate like elements.

### DETAILED DESCRIPTION OF ILLUSTRATIVE EMBODIMENTS

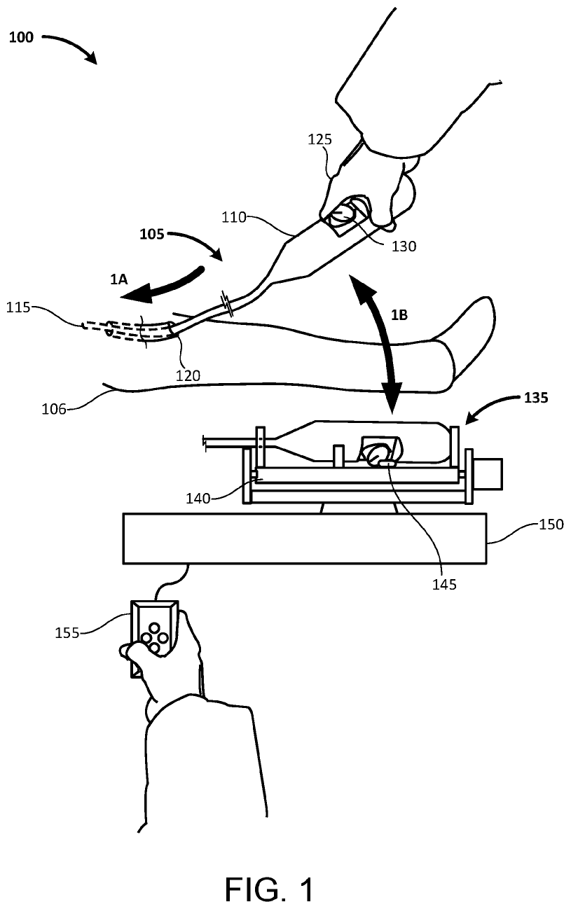

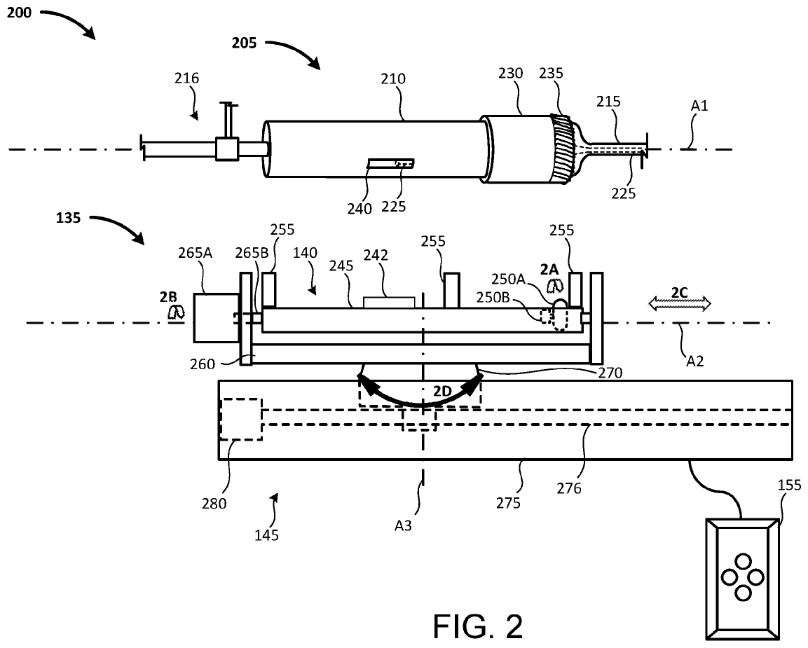

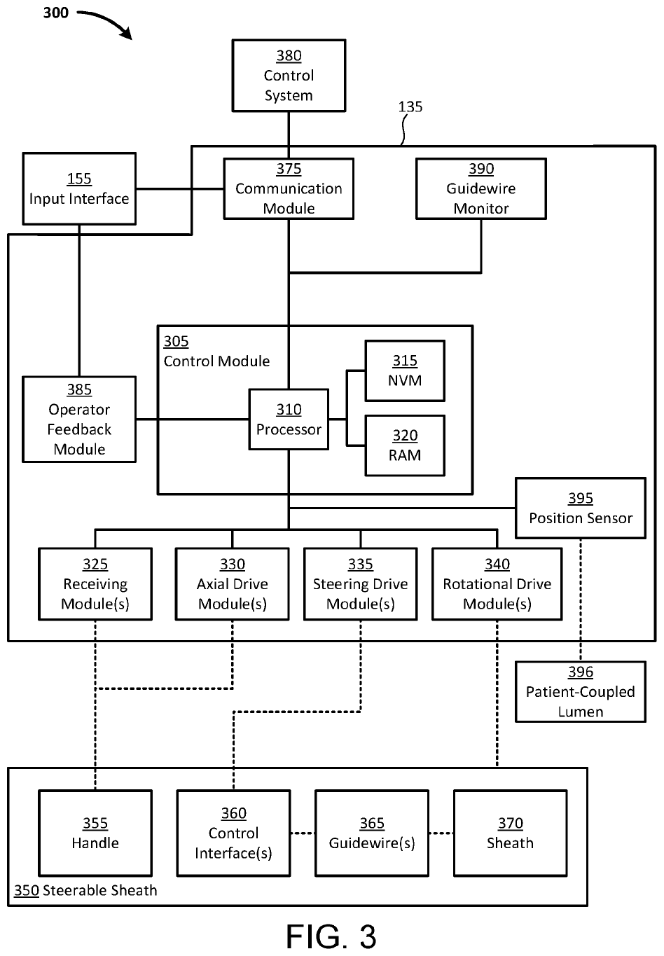

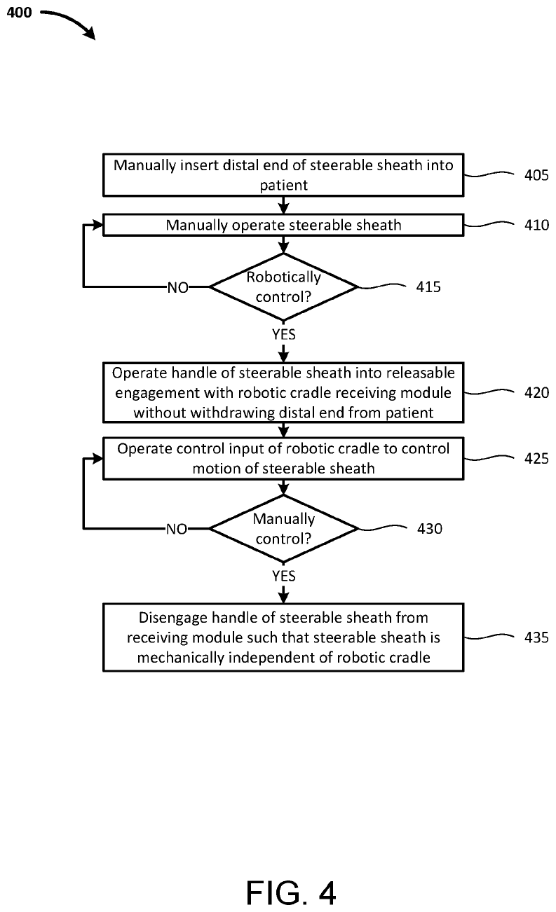

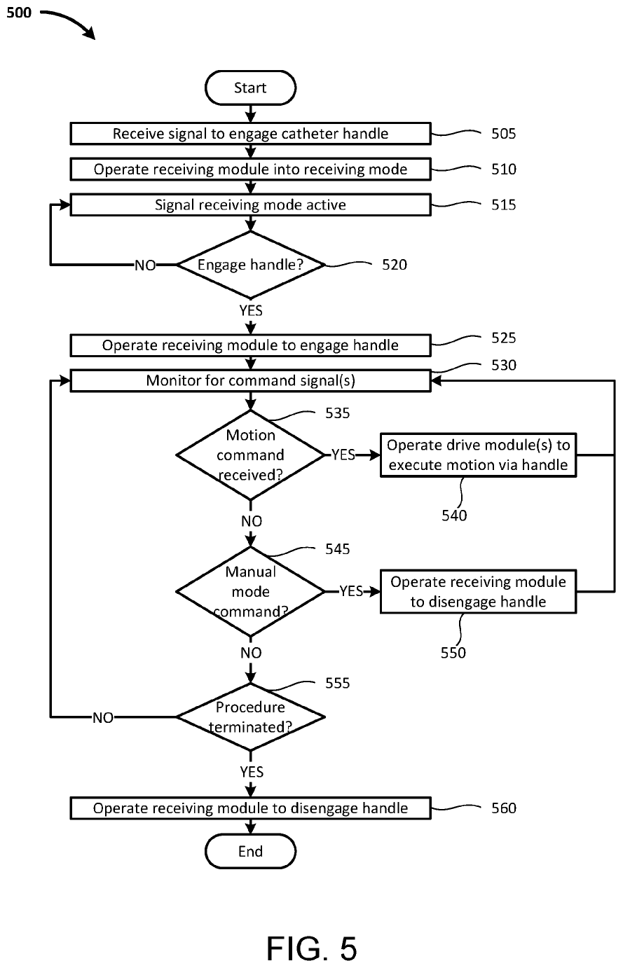

To aid understanding, this document is organized as follows. First, to help introduce discussion of various embodiments, a robotic catheter cradle (RCC) system is introduced with reference to FIG. 1. Second, that introduction leads into a description with reference to FIGS. 2-3 of some exemplary embodiments of RCCs. Third, with reference to FIG. 4, an exemplary method of operating a catheter using a selectively independent RCC is described. Fourth, with reference to FIG. 5, the discussion turns to an exemplary method of automatic operation of an RCC, including between a robotic mode and a mechanically independent manual mode. Finally, the document discusses further embodiments, exemplary applications and aspects relating to RCCs.

FIG. 1 depicts an exemplary robotic catheter cradle (RCC) employed in an illustrative use-case scenario in which a human operator selectively operates a steerable sheath between a robotically operated mode and a manual mode that permits operation mechanically independent of the RCC. In a depicted scenario 100, a steerable sheath 105 includes a handle 110. A steerable sheath extends from the handle 110 to a distal end of the conduit 115. As depicted, the distal end of the conduit 115 is introduced (motion “1A”) into a patient 106 through a lumen of a dilator 120. The dilator 120 may, for example, be sutured to the patient 106 (e.g., into a blood vessel). In some implementations, the dilator 120 may, for example, be an introducer. The operator 125 manipulates the handle 110, by way of example and not limitation, to advance the distal end of the conduit 115 (e.g., relative to the dilator 120). The operator 125 may operate an interface 130 (e.g., a rotatable knob as depicted) to steer the distal end of the conduit 115. For example, the interface 130 may be operably coupled to guidewire(s) configured to controllably deflect the distal end of the conduit 115.

In the depicted example, once the distal end of the conduit 115 is introduced into the patient 106 (e.g., after the operator 125 has manually operated the steerable sheath 105 such that the distal end of the conduit 115 has reached a desired region), the operator 125 operates (motion “1B”) the steerable sheath 105 into releasable engagement with a robotic catheter cradle 135 (e.g., a robotic catheter ‘cradle’). For example, the operator 125 may releasably engage the handle 110 in a cradle 140 of the robotic catheter cradle 135. The robotic catheter cradle 135 is provided with a steering drive member 145 configured to engage the interface 130. Accordingly, the robotic catheter cradle 135 may robotically control, for example, deflection of the distal end of the conduit 115.

The cradle 140 may, for example, be rotatably mounted. Accordingly, for example, the robotic catheter cradle 135 may robotically control rotation of the conduit 115 (e.g., via rotation of the handle 110). The cradle 140 is translatably coupled to a base 150. The base 150 may, for example, robotically control advancement and/or retraction of the steerable sheath 105 (e.g., via translation of the handle 110).

As depicted, the operator 125 may control the robotic catheter cradle 135 via a human-machine interface 155. In the depicted example, the human-machine interface 155 includes a 4-button keypad interface (e.g., providing various motions in response to operation of the interface by the operator 125. In the depicted example, the human-machine interface 155 is operably connected to the lower base 275.

The operator 125 may, for example, steer the steerable sheath 105 via the robotic catheter cradle 135 in a robotic mode. In some examples, the operator 125 may subsequently determine further manual intervention is necessary and/or desirable. For example, the operator 125 may desire to apply a specific orientation and/or manipulation to the steerable sheath 105 (e.g., bending the conduit 115 at an angle against the leg of the patient 106) that is not possible and/or feasible in the robotic mode. Accordingly, the operator 125 may operate the steerable sheath 105 back into a manual mode (motion “1B”), without withdrawing the conduit 115 from the patient or disconnecting the conduit 115 from the handle 110. In the manual mode, the steerable sheath 105 may be mechanically independent from the robotic catheter cradle 135. For example, in the manual mode, the steerable sheath 105 may be substantially entirely mechanically disengaged from the robotic catheter cradle 135, including the handle 110 and the conduit 115 (e.g., including from the distal end of the handle 110 to the point at which the conduit 115 enters the patient 106). The dilator 120 may, for example, be mechanically independent of the robotic catheter cradle 135.

Such embodiments may, for example, enable the operator 125 to rapidly and/or easily operate the steerable sheath 105 into the (mechanically) independent manual mode. For example, the operator 125 may operate the steerable sheath 105 into the manual mode by disengaging the handle 110 from the cradle 140. In some embodiments, by way of example and not limitation, no changes may be required to the steerable sheath 105. In some embodiments, the handle 110 may, for example, be mechanically removed from the cradle 140 (e.g., ‘snapped out’) by the operator 125.

Mechanical independence of the steerable sheath 105 from the robotic catheter cradle 135 may, for example, advantageously increase patient safety. For example, a malfunction of the robotic catheter cradle 135 (e.g., a malfunctioning sensor, a malfunctioning actuator, a malfunctioning controller) may endanger the patient 106. Embodiments which allow the operator 125 to rapidly operate the steerable sheath 105 into the manual mode (e.g., without disengaging the conduit 115 from the handle 110 and/or withdrawing the conduit 115 from the patient 106) may advantageously enable the operator 125 to prevent or reduce damage to the patient from a malfunction.

Mechanical independence of the steerable sheath 105 from the robotic catheter cradle 135 may, for example, advantageously increase mechanical flexibility. For example, a unique aspect of a patient's physiology may unexpectedly interfere with reaching a desired target. A specific mechanical inducement may be needed that is not possible and/or feasible to achieve robotically. For example, an experienced physician may wish to apply, by way of example and not limitation, a specific rotation, flexure, force, compound mechanic (e.g., flexure, pressure, twisting), or some combination thereof. A physician may, for example, wish to receive tactile feedback by manually holding the handle 110 during manipulation (e.g., during a particularly sensitive and/or unusual portion of a procedure). Accordingly, embodiments which allow the operator 125 to freely operate the steerable sheath 105 between a robotic mode using the robotic catheter cradle 135 and a manual mode (mechanically) independent of the robotic catheter cradle 135 may advantageously achieve an operator-specific ‘ideal’ mixture of advantages of robotic and manual manipulation.

In some embodiments, such as depicted, the robotic catheter cradle 135 may be mechanically decoupled from (e.g., independent of) an introducer and/or dilator. Such embodiments may, for example, advantageously allow the operator 125 to gain full manual control of the handle 110 and the conduit 115. For example, various embodiments may advantageously avoid a portion of the conduit 115 from being ‘trapped’ by a robotic introducer and/or aperture through a robotic system.

As an illustrative example, embodiments (e.g., as depicted in FIG. 1) may, for example, provide an operator the ability to transition from manual to robotic control with a steerable sheath handle and handle stand. Such embodiments may, by way of example and not limitation, allow an operator to manually use a steerable sheath 105 (e.g., by holding handle 110 and (operating) interface 130) in a method that is familiar to get a tip of the conduit 115 close to a final target position. The operator may then, for example, place the handle 110 in the robotic catheter cradle 135. Accordingly, the robotic catheter cradle 135 may advantageously free the operator such as, by way of example and not limitation, to manipulate other devices, to fine tune final position, to change position of the sheath, or some combination thereof. Various embodiments may, for example, advantageously allow an operator to robotically adjust the catheter during fluoroscopy while avoiding a radiation beam, thereby advantageously minimizing radiation exposure of the operator.

FIG. 2 depicts an exemplary RCC and steerable sheath handle. In a robotic catheter system 200, a steerable sheath 205 of a handheld catheter system is configured to be removably mounted to a robotic catheter cradle 135. The robotic catheter cradle 135 may, for example, include a controller (not shown), one or more communication modules (not shown), or some combination thereof. For example, the robotic catheter cradle 135 may be operably coupled to and/or include elements such as are disclosed at least with reference to FIG. 3 herein.

The steerable sheath 205 includes a control module 230. The control module 230 is provided with engagement elements 235. The control module 230 may, for example, be operably coupled to control an orientation and/or geometry of a steerable conduit 215. The steerable conduit 215 is fluidly coupled to a source conduit 216. As depicted, the source conduit 216 may include a 3-way valve and/or multiple source conduits.

For example, as depicted, the control module 230 may be configured as an input interface. An input module (not shown) may generate an input signal in response to operation of the control module 230. For example, the input module may generate an input signal(s) in response to rotation of the control module 230 relative to the steerable sheath 205 about a longitudinal axis A1. Corresponding control members 225 may, for example, be actuated (e.g., translated substantially parallel to A1) to induce a desired deflection in the steerable conduit 215.

In some embodiments, by way of example and not limitation, the corresponding control members 225 may be guidewires. The guidewires may, for example, be selectively and independently actuated in response to operation of the control module 230 (e.g., via the engagement elements 235, directly via the ‘collar’ of the control module 230). In some embodiments, the guidewires may, by way of example and not limitation, be automatically tensioned. Exemplary catheters and/or tensioning systems are disclosed at least with reference to FIGS. 1-11 (e.g., particularly with respect to a robotically manipulated catheter in FIG. 9) of U.S. application Ser. No. 17/453,988, filed by John Pocrnich, et al., the entire contents of which are incorporated herein by reference.

In the depicted example, the handle 210 is provided with a window 240. The window 240 may, for example, be configured for visual monitoring of a status of one or more of the corresponding control members 225. For example, a position of a ferrule on the corresponding control members 225 may, for example, be viewed through the window 240. For example, an operator may advantageously visually determine a status (e.g., orientation, position) of the distal end of the steerable conduit 215.

The robotic catheter cradle 135 is provided with a cradle 140 (e.g., a receiving module). The cradle 140 includes a carriage 245 configured to receive the steerable sheath 205. A steering drive module includes a drive member 250A and an actuator 250B. The drive member 250A may be configured to releasably engage the engagement elements 235. In the depicted example, the drive member 250A is driven by the actuator 250B (e.g., an electric motor). The actuator 250B may, for example, be operably coupled to and/or responsive to a controller.

The carriage 245 includes a coupling member 255 which may be configured, for example, to releasably couple to the steerable sheath 205 (e.g., behind the control module 230). Accordingly, the steerable sheath 205 may be releasably axially and rotationally coupled to the carriage 245. In some embodiments, by way of example and not limitation, the coupling member 255 may be spring-loaded. In some embodiments, the coupling member 255 may be manually operable (e.g., moved between a locked mode and an open mode). In some embodiments, the coupling member 255 may ‘clamp.’ For example, in some embodiments, a moving jaw(s) may be operated to releasably couple to the handle 210. In some embodiments, the coupling member 255 may, for example, be manually operated. In some embodiments, the coupling member 255 may, for example, be robotically operated. In some embodiments, multiple coupling members may be provided. For example, a distal and/or proximal member, as depicted, may be coupling members.

The control module 230 may be rotatable about A1 relative to the steerable sheath 205 and the carriage 245. When the steerable sheath 205 is releasably coupled into the carriage 245 (e.g., in a robotic mode), the engagement elements 235 may be held engaged against the drive member 250A. A pattern on the engagement elements 235 may, for example, be complementary to a pattern (not shown) on the drive member 250A. The drive member 250A may rotate, as shown by motion “2A” (e.g., by the actuator 250B, in response to a command from an operator), thereby inducing rotation about A1 of the control module 230 via the engagement elements 235 (e.g., having a gear-tooth pattern). Accordingly, the control module 230 may rotate relative to the steerable sheath 205, thereby inducing a deflection (e.g., commanded via the human-machine interface 155) in the steerable conduit 215. A steerable tip (e.g., a distal end) of the steerable conduit 215 may thereby be advantageously controlled.

As depicted, the carriage 245 is further provided with a (pull wire location) monitor 242 (e.g., an optical sensor, a magnetic sensor). The monitor 242 may be configured to monitor the window 240 of the steerable sheath 105 when the steerable sheath 105 is releasably coupled within the carriage 245. The robotic catheter cradle 135 may, for example, be operably coupled to an automatic feedback control circuit and/or manual feedback configured to provide adjustable operation of a pull wire in the steerable sheath to achieve and/or maintain a given configuration of a delivery shaft (e.g., conduit 115). For example, in various embodiments the monitor 242 may be configured to sense an end(s) (e.g., a ferrule(s)) of a pull wire in the handle 110 via the window 240. A controller may, for example, be configured to calculate the bend in the tip based on signals from the monitor 242. One or more correction circuits and associated mechanism may be configured to correct the bend as catheters are passed through the (flexible) conduit 115. Various such embodiments may, by way of example and not limitation, be configured as discussed at least in relation to FIGS. 4A-4C of U.S. application Ser. No. 17/453,988, filed by John Pocrnich, et al., the entire contents of which are incorporated herein by reference.

The carriage 245 may be rotatably coupled to a frame 260. For example, the carriage 245 may be configured to rotate (motion “2B”) relative to the frame 260 about a longitudinal axis A2 of the carriage 245. A carriage actuator 265A is operably coupled to the carriage 245. The carriage actuator 265A is operably coupled to rotate a shaft 265B such that operation of the carriage actuator 265A may induce rotation of the frame 260 about the longitudinal axis of the carriage 245. The carriage actuator 265A may, for example, include a motor. The carriage 245 may, for example, be operated by a controller (e.g., in response to commands of an operator). Accordingly, (controlled) rotation of the steerable sheath 205 may be advantageously induced about A2 and/or an axis substantially parallel to A2 when the steerable sheath 205 is releasably coupled to the carriage 245.

The frame 260 is coupled to an upper base 270. For example, the frame 260 may be slidably and/or rotatably coupled to the upper base 270.

The upper base 270 is coupled to a lower base 275. The upper base 270 may, for example, be rotatably and/or slidably coupled to the lower base 275 (e.g., by a threaded collar). As depicted, the upper base 270 is movably coupled to a drive member 276 (e.g., a threaded rod, a hydraulic cylinder, a pneumatic cylinder). An actuator 280 (e.g., a motor, a fluid pressure source, a fluid valve) is configured to advance and/or retract the upper base 270 along A2 relative to the lower base 275. Accordingly, the actuator 280 may controllably advance and/or retract the frame 260 along A2 relative to the upper base 270 (motion “2C”). In some embodiments, the actuator 280 and/or the drive member 276 may be configured as a linear slide (e.g., telescoping, rack and pinion, linear actuator) to drive the frame 260 and/or the upper base 270 relative to the lower base 275.

Accordingly, an operator may, for example, advantageously precisely advance/retract the steerable conduit 215 (e.g., by operating the frame 260), rotate the steerable conduit 215 (e.g., by rotating the carriage 245), steer the steerable conduit 215 (e.g., by rotating the drive member 250A), or some combination thereof. An operator may advantageously release the steerable sheath 205 from the robotic catheter cradle 135 while maintaining it substantially fixed in a desired configuration (e.g., by operating into a manual mode without having to withdraw and/or decouple the steerable conduit 215 from the handle 210). Various embodiments may, in a robotic mode, passively maintain a desired configuration (e.g., by resisting movement) and/or actively maintain a desired configuration (e.g., by operating one or more actuators to recover a desired configuration and/or resist motion).

An actuator (not shown) may be configured to rotate the frame 260 about an axis A3 relative to the upper base 270 (motion “2D”). In some embodiments, an actuator (not shown) may, for example, slew the frame 260 (e.g., along a track, not shown) relative to the lower base 275. Such embodiments may, for example, advantageously enable the handle 210 to be angled relative to the steerable conduit 215 without moving the proximal end (closest to the handle 210) of the steerable conduit 215 to be translated off of A1. In some embodiments, the upper base 270 may be located (e.g., moved forward towards the drive member 250A) such that A3 is substantially aligned vertically with a proximal end of the steerable conduit 215.

In some embodiments, for example, the frame 260 may rotate about A3 relative to the upper base 270. The upper base 270 may, by way of example and not limitation, translate along A2 relative to the lower base 275. In some embodiments, for example, the frame 260 may translate along A2 relative to the upper base 270. The upper base 270 may, for example, rotate about A3 relative to the lower base 275.

In various embodiments the upper base 270 may, for example, include a turret configured to allow horizontal rotation (e.g., slew and/or pitch) of the frame 260 relative to the lower base 275. Accordingly, various embodiments may provide a steerable robotic catheter (system) which (a) makes it easier for an operator (e.g., a physician) to navigate to a desired location, and/or (b) maintains the location.

The lower base 275 is operably coupled to a human-machine interface 155. As depicted, the human-machine interface 155 is in wired electrical communication with the lower base 275. The human-machine interface 155 may, for example, be coupled to a controller.

In such embodiments, the human-machine interface 155 may, by way of example and not limitation, be wirelessly coupled to the control module 305 (e.g., in the robotic catheter cradle 135) disclosed at least with reference to FIG. 3. The human-machine interface 155 may, for example, be a dedicated HMI. The human-machine interface 155 may, for example, be a multipurpose HMI (e.g., a mobile computing device). In some embodiments, the human-machine interface 155 may, for example, include a visual feedback (e.g., a display screen), tactile (e.g., haptic) feedback mechanism(s), or some combination thereof. The human-machine interface 155 may, for example, transduce (mechanical) inputs from a user into signals provided to the control module 305. The control module 305 may, for example, generate signals to operate the various actuators (e.g., actuator 250B, carriage actuator 265A, actuator of the drive member 250A, actuator of the frame 260, actuator of the upper base 270, actuator of the handle 210). Accordingly, an operator (e.g., a physician) may advantageously operate the steerable sheath 205 via the human-machine interface 155.

In some embodiments, the human-machine interface 155 may have multiple user inputs (e.g., button(s), joystick(s), switches, touchscreen, accelerometer, gyroscope) configured to transduce physical motion of an operator into control outputs to one or more circuits in the robotic catheter cradle 135. Control outputs may be configured to cause operation of the frame 260 relative to the lower base 275 (e.g., by operation of a linear motor in the lower base 275), rotation of the drive member 250A (e.g., by operation of a motor within the carriage 245), rotation of the carriage 245 (e.g., by operation of the carriage actuator 265A), or some combination thereof. In various embodiments the human-machine interface 155 may, for example, be wired to the robotic catheter cradle 135 and/or be wirelessly connected thereto. Various embodiments may, for example, advantageously allow an operator to operate the catheter from a remote location (e.g., in response to one or more displays).

Various embodiments, such as depicted, may advantageously provide multiple (e.g., 2, 3, 3+) degrees of freedom of the steerable sheath 205 (e.g., motion 2B, 2C, 2D). Various embodiments may advantageously operate the steerable conduit 215 along at least one additional degree of freedom (e.g., 2, 3 degrees of freedom). Accordingly, (robotic) control of the steerable sheath 205 may advantageously provide selectable (precise) control over a steerable conduit 215 and associated steerable tip.

FIG. 3 depicts an exemplary block diagram of an exemplary RCC and steerable sheath handle. An exemplary RCC system 300 includes a robotic catheter cradle 135. The robotic catheter cradle 135 includes a control module 305. The control module 305 is provided with a processor 310, a non-volatile memory module 315 (“NVM”), and a random-access memory module 320 (“RAM”). The non-volatile memory module 315 is electrically coupled to the processor 310. The processor 310 is electrically coupled to the random-access memory module 320. The non-volatile memory module 315 may, for example, store one or more programs of instruction which, when executed by the processor 310, perform operations to (robotically) control a steerable sheath.

The control module 305 is operably (e.g., electrically and/or mechanically) coupled to a receiving module(s) 325 (e.g., the cradle 140). The control module 305 is operably coupled to an axial drive module 330 (e.g., including the upper base 270, drive member 276, and/or actuator 280). The control module 305 is operably coupled to a steering drive module(s) 335 (e.g., including the drive member 250A and/or the 250B). The control module 305 is operably coupled to a rotational drive module(s) 340 (e.g., including the carriage actuator 265A, the shaft 265B, and/or the carriage 245).

The robotic catheter cradle 135 is configured, in the depicted example, to (selectively) robotically operate a steerable sheath 350 (e.g., the steerable sheath 105, the steerable sheath 205). The receiving module(s) 325 are releasably operably (e.g., mechanically) coupled to a handle 355 (e.g., the handle 110, the handle 210). The receiving module(s) 325 may releasably couple to the handle 355, for example, in response to operation of the steerable sheath 350 into a robotic mode.

The axial drive module(s) 330 is operably (e.g., mechanically) coupled to the handle 355. For example, the axial drive module(s) 330 may be mechanically coupled to the receiving module(s) 325. The axial drive module(s) 330 may translate (e.g., advance, retract) the handle 355 in response to command signals.

The steering drive module(s) 335 are operably (e.g., mechanically, electrically, magnetically) coupled to control interface(s) 360. At least one of the interface(s) 360 are operably (e.g., mechanically) coupled to a guidewire(s) 365 (e.g., the control members 225). The guidewires 365 are operably (e.g., mechanically) coupled to a (flexible) sheath 370 (e.g., the steerable conduit 215) Accordingly, the steering drive module(s) 335 may, for example, operate the interface(s) 360 to controllably deflect a tip of the sheath 370. For example, in some embodiments, the steering drive module(s) may be configured to operate one or more interface(s) 360 configured to be operated by a human operator (e.g., knob, slider, lever) when the steerable sheath 350 is engaged in the robotic catheter cradle 135 in the robotic mode.

The control module 305 is further operably coupled (e.g., electrically and/or mechanically) to a communication module 375. The communication module 375 may, for example, be configured to communicate (e.g., receive and/or transmit signals) between the control module 305 and one or more external devices. The communication module 375 may, for example, communicate with an (external) HMI, with one or more portable devices (e.g., smartphones, tablets, remote control units), servers, computing devices, other medical tools (e.g., robotic catheter system), or some combination thereof. In various embodiments the communication module 375 may, for example, be provided with wired and/or wireless (e.g., Bluetooth, Wi-Fi) communication ports. Ports may, for example, be permanently connected. Ports may, for example, be pluggably connected (e.g., USB, HDMI, RJ45).

The communication module 375 is operably coupled to a control system 380. For example, the control system 380 may provide (predetermined) motion commands The communication module 375 is operably coupled to the human-machine interface 155. The human-machine interface 155 may, for example, generate signals corresponding to motion commands from an operator.

In various embodiments, the human-machine interface 155 may, by way of example and not limitation, include a button, switch, knob, lever, slider, touch screen, display, mobile device (e.g., smartphone, laptop, tablet), or some combination thereof. For example, a human-machine interface 155 may include a remote-control interface. The human-machine interface 155 may, for example, be provided by an app running on a computing device (such as a mobile device and/or computing device).

The human-machine interface 155 may, for example, be configured to transduce input(s) from an operator into command and/or feedback signals to the control module 305. The human-machine interface 155 may, for example, be configured to generate feedback signals (e.g., visual, audio, tactile) for an operator in response to command signals from the control module 305. For example, in some embodiments the human-machine interface 155 may provide a visual display of formation of a unitary stoma closure (e.g., from an optical sensor such as a camera at a distal end of the sheath 370). In some embodiments the human-machine interface 155 may, for example, receive commands from the operator (e.g., related to steering the sheath 370, advancing and/or withdrawing the sheath 370, operating the drive module(s)).

The control module 305 is operably coupled to an operator feedback module 385. The operator feedback module 385 is operably coupled to the human-machine interface 155. For example, the operator feedback module 385 may generate, in response to signals from the processor 310 (e.g., corresponding to feedback from a drive module, a sensor, an actuator), feedback signals. The human-machine interface 155 may provide the feedback, in response to the feedback signals, to an operator(s). For example, the feedback may be visual (e.g., lights, text, icons, graphics). The feedback may, for example, be audible. For example, the feedback may be haptic (e.g., vibration, force/resistance, motion). In some embodiments, for example, substantially real-time feedback may be provided to the operator.

In the depicted example, the robotic catheter cradle 135 includes a guidewire monitor 390 (e.g., monitor 242). The guidewire monitor 390 may, for example, monitor a deflection status of a distal end of the sheath 370 (e.g., via the window 240). The guidewire monitor 390 may, for example, generate feedback signal(s) to the control module 305. The control module 305 may, for example, control one or more drive module (e.g., the axial drive module(s) 330, the steering drive module(s) 335, the rotational drive module(s) 340) in response to signal(s) form the guidewire monitor 390.

In the depicted example, the processor 310 is operably coupled to a position sensor 395. The position sensor 395 may, for example, generate a signal corresponding to a distance. The position sensor 395 may, for example, generate a signal corresponding to an orientation (e.g., an angle). In some embodiments, the position sensor 395 may, for example, be a position sensing module including multiple sensors and/or computing module(s). The position sensor 395 is operably coupled to a patient-coupled lumen 396 (e.g., the dilator 120). For example, the position sensor 395 may be mechanically coupled to the patient-coupled lumen 396. In some embodiments, the position sensor 395 may, by way of example and not limitation, be optically coupled to the 396. The position sensor 395 may, for example, be electronically coupled to the patient-coupled lumen 396.

The control module 305 may, by way of example and not limitation, further operably (e.g., electrically and/or mechanically) be coupled to one or more sensors (e.g., the monitor 242) and/or actuators. For example, one or more sensors may be configured to monitor a conduit (e.g., steerable conduit 215). In some embodiments, one or more sensors may be configured to monitor a delivery target. Accordingly, the control module 305 may, by way of example and not limitation, advantageously receive feedback via a sensor regarding a conduit (e.g., steerable conduit 215) and/or a delivery target.

The control module 305 may, by way of example and not limitation, be further operably (e.g., electrically and/or mechanically) coupled to an actuator (e.g., one or more actuators). An actuator may be configured to act upon and/or be a component of a drive module. In various embodiments an actuator may, by way of example and not limitation, operate other modules. Other modules may include, by way of example and not limitation, a phase transition inducement module (e.g., dispensing a phase transition inducing component, operating a light shutter and/or electrical contact, operating a thermal element).

In some embodiments a control module may be operably coupled to an end module on a conduit, such as the steerable conduit 215. An end module may, for example, include a piercing tool(s). An end module may, for example, include a cutting tool(s). An end module may, for example, include a suture tool(s). An end module may, for example, include a sensor such as an optical sensor).

FIG. 4 is a flowchart illustrating an exemplary method of introducing and operating a steerable sheath in a robotic mode and a manual mode mechanically independent of an RCC. In a method 400, a distal end of a steerable sheath (e.g., conduit 115, steerable conduit 215, sheath 370) is manually inserted into a patient (e.g., patient 106) in a step 405. The steerable sheath is manually operated (e.g., by a handle of the steerable sheath) in a step 410. Once it is determined, at a decision point 415 that the steerable sheath should be robotically controlled (e.g., in response to reaching a predetermined location in the patient and/or milestone in the procedure, in response to operator fatigue), then the handle of the steerable sheath is operated, in a step 420, into releasable engagement with a robotic cradle receiving module (e.g., cradle 140) without withdrawing the distal end from the patient.

A control input (e.g., HMI) of the robotic cradle is operated, in a step 425, to control motion of the steerable sheath. If it is determined, in a decision point 430, that manual control should be resumed, then the handle of the steerable sheath is disengaged from the receiving module in a step 435. Accordingly, the steerable sheath may be selectively operated into mechanical independence (e.g., mechanical isolation from) the robotic cradle at will and without withdrawing the distal end from the patient.

The method 400 may, for example, be performed by an operator (e.g., the operator 125). For example, the operator may be a physician. In some embodiments, at least part of the method 400 may be performed by a robotic controller (e.g., step 420, step 425).

FIG. 5 is a flowchart illustrating an exemplary method of automatic control of an RCC in a robotic catheter control mode and a mechanically independent manual mode. A method 500 may, by way of example and not limitation, be automatically performed by a controller (e.g., the control module 305 disclosed at least with reference to FIG. 3). The method 500 may, for example, be performed in response to the step 425 of the method 400 disclosed at least with reference to FIG. 4.

In the method 500, a signal(s) is received to engage a catheter handle (e.g., a steerable sheath handle) in a step 505. The signal(s) may, for example, be automatically generated in response to the handle being operated into a robotic mode. The signal may, for example, be generated when a specific input(s) (e.g., button, lever, key sequence) is operated.

The receiving module is operated into a receiving mode in a step 510. For example, an actuator may position and/or orient a receiving module for receiving the catheter handle. As an illustrative example, a jaw may open (e.g., slidably, pivotably). In the depicted example, a signal is then generated, in a step 515, that the receiving mode is active. The signal may, for example, cause a visual, audio, and/or haptic indicia to be generated for an operator.

Once it is determined, in a decision point 520, that the handle is engaged (e.g., positioned in a predetermined orientation) in the receiving module, then the receiving module is operated to engage the handle in a step 525. For example, decision point 520 may be automatically determined when the handle is placed into a robotic cradle. The decision point 520 may, for example, be determined when the handle is engaged in the robotic cradle and a robotic control input signal(s) is received (e.g., in response to operation of a (dedicated) input) from the operator.

The method 500 (e.g., the control module 305) monitors for a command signal(s) in a step 530. If a command signal is determined, in a decision point 535, to correspond to a motion command, then corresponding drive module(s) (e.g., axial drive module(s) 330, steering module(s) 335, rotational drive module(s) 340) are operated, in a step 540, to execute motion via the handle engaged in the receiving module.

If a command signal is determined to correspond to a manual mode command, in a decision point 545, then the receiving module (e.g., receiving module(s) 325) is operated, in a step 550, to disengage the handle. An operator may, for example, remove the handle from the receiving module.

In the depicted example, if a command signal is determined, in a decision point 555, to correspond to a termination of a current procedure, then the receiving module is operated (in a step 560) to disengage the handle, and the method 500 ends.

In some embodiments, a robotic catheter cradle may be configured for an operator to engage or disengage a handle without waiting for the robotic catheter cradle to respond and/or act. For example, the handle may be ‘snapped’ out and/or a receiving module may be automatically moved into an engaged or disengaged state. The receiving module may, for example, automatically detect presence of the handle (e.g., via a proximity sensor and/or switch, optical sensor) and automatically self-operate into an appropriate mode in response.

Although various embodiments have been described with reference to the figures, other embodiments are possible. For example, although exemplary systems have been described with reference to FIGS. 1-5 other implementations may be deployed in other industrial, scientific, medical, commercial, and/or residential applications.

Various embodiments may advantageously provide a steerable catheter (system) which makes a steerable catheter efficient to use. For example, various embodiments may provide steerable catheters which maintain a desired position. Accordingly, procedure times may be decreased, which may advantageously reduce healthcare expenditures. Various embodiments may advantageously reduce exposure of a clinical team and/or patient to unnecessary radiation (e.g., by allowing remote operation of the catheter, reducing procedure time). Various embodiments may, for example, advantageously replace bulky and/or robotic systems which physicians have been slow to adopt.

In various embodiments, the robotic platform may be designed to be lightweight and/or to rest on a patient's leg. For example, a robotic catheter cradle (e.g., robotic catheter cradle 135) may be configured to be coupled to a patient's body (e.g., strapped). In some embodiments, the robotic platform may be coupled to a table, cart, and/or mechanical arm. In some such embodiments, a positioning controller may reference position control off of a predetermined reference point (e.g., a distal end of a dilator and/or introducer). The positioning controller may interpret commanded motion and/or make motion adjustments as a function of the reference point. The controller may, for example, automatically adjust motion (e.g., axial, deflection, and/or rotational motion) based off of the predetermined reference point.

In various embodiments, the steerable sheath 105 (e.g., the handle 110 and/or the conduit 115) may be sterile and disposable. The robotic platform may, for example, be re-sterilizable and re-usable.

In various embodiments, a base (e.g., the lower base 275) may include, by way of example and not limitation, a battery (or other suitable power supply) and electronics (e.g., one or more control circuits). In various embodiments a surface (e.g., a bottom surface) of a base (e.g., the lower base 275) may be shaped to fit a patient's leg (e.g., a concave surface configured to fit over the curvature of a patient's leg).

In some embodiments, by way of example and not limitation, a cradle (e.g., including the frame 260) may angle relative to a base (e.g., the lower base 275) in a plane defined by a longitudinal axis of the cradle and the base (e.g., axis A2 and a longitudinal axis of the lower base 275). Such embodiments may, for example, advantageously enable the robotic cradle to maintain and/or achieve a desired angle of a conduit (e.g., the steerable conduit 215) relative to a patient. Some such embodiments may, by way of example and not limitation, automatically adjust in such a plane to maintain a commanded orientation relative to a predetermined reference(s) (e.g., coupled to the patient).

In various embodiments, by way of example and not limitation, a robotic catheter cradle (e.g., the robotic catheter cradle 135) and/or steerable sheath (e.g., the steerable sheath 105) may be configured for use in an atrial fibrillation procedure. For example, a physician may operate the steerable sheath 105 (e.g., using robotic catheter cradle 135 via human-machine interface 155) to create a transseptal puncture and provide a path for an ablation catheter. In an exemplary method, a clinician may introduce a disposable steerable delivery conduit (e.g., the conduit 115) into the patient and position it such that a tip is at a septal wall that separates a right and a left atrium. The clinician may then snap the handle 110 of the steerable sheath 105 into the carriage 245. Fine adjustments to tip deflection, tip advancement and/or tip rotation may then be made using the robotic system (e.g., using human-machine interface 155). The sheath may, for example, contain a dilator. The dilator may then be removed and a needle, such as a Brockenbrough needle, may be advanced.

The needle may, for example, have a different stiffness than the dilator. Sensors may, by way of example and not limitation, detect a corresponding change in tip deflection (e.g., by monitor 242 of (monitoring) window 240) due to the stiffness of the needle, and operate one or more actuators to correct the deflection and keep the tip in the correct location (e.g., by (operating) drive member 250A). Once the puncture is made, the sheath tip can be advanced into the left atrium, the needle removed, and an ablation catheter passed through the conduit 115. The advancement can be done, for example, using the robotic system (e.g., by an actuator in robotic catheter cradle 135). Again, the tip position may be maintained as a different catheter is passed through the conduit 115. The robotic system may, for example, be used to assist the ablation catheter to access various parts of the left atrium during the procedure. At any time, the clinician can remove the sheath from the robotic stand and resume manual control.

In some embodiments, any type of steerable catheter may be used. Steerable shafts which may be used include, by way of example and not limitation, electrophysiology. For example, an RCC system may be advantageously configured for steerable ablation, intracardiac echo (ICE), and/or mapping catheter(s). Various embodiments may be applied and/or configured for use in procedures such as, by way of example and not limitation, structural heart applications. Various embodiments may, for example, be applied and/or configured for use in pulmonary (e.g., lung) procedures. Various embodiments may, for example, be applied and/or configured for use in gastrointestinal procedures.

Various embodiments may be configured to operate a predetermined sequence of operations including, by way of example and not limitation, sled advancement/retraction, sled rotation, cradle rotation, drive mechanism operation, catheter advancement, or some combination thereof. For example, a predetermined sequence of operations may be associated with one or more operations. A physician may, for example, predetermined a sequence of operations based on a physiological model (e.g., a standard model and/or a patient-specific model such as generated by magnetic resonance, computed tomography, ultrasound). The sequence(s) of operations may, for example, be stored in one or more data stores. The sequence(s) may then be retrieved and implemented. Various embodiments may, for example, store at least some portion of the sequence of operations in local memory in the steerable sheath 105 and/or robotic catheter cradle 135, in a remote controller (e.g., computer, mobile device, interactive processor-based controller), or some combination thereof. In various embodiments the operations may be executed by a local processor and/or a remote processor (e.g., configured to send control outputs to the robotic catheter cradle 135).

Various embodiments may, for example, omit a monitoring window (e.g., window 240). Various embodiments may include a monitoring means such as, by way of example and not limitation, a position sensor (e.g., a linear scale), displacement sensor, force sensor, rotation sensor, angle sensor, or some combination thereof. Sensor(s) may, for example, monitor a position and/or orientation of a frame (e.g., frame 260), a carriage (e.g., carriage 245), a pull wire (e.g., control members 225), a drive member (e.g., drive member 250A), an interface (e.g., interface 130), a handle (e.g., handle 110), a sheath (e.g., conduit 115, a distal tip of a conduit), or some combination thereof. A controller(s) may receive input(s) from the sensor(s). In response to the input(s), the controller(s) may, for example, provide feedback to a user and/or drive one or more actuators to maintain and/or achieve a desired configuration. Various embodiments may, by way of example and not limitation, include sensors, feedback, and/or control circuits (e.g., of a pull wire).

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) battery, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device, such as a CRT (cathode ray tube) or LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In an illustrative aspect, an apparatus may include a robotic catheter cradle. The robotic catheter cradle may include a receiving module extending along a longitudinal axis and configured to releasably receive at least a handle of a steerable sheath after a distal end of the steerable sheath has been manually introduced into a patient independent of the receiving module. The apparatus may include a steering drive module operably coupled to the robotic catheter cradle to robotically operate at least one interface of the handle to selectively actuate at least one guidewire of the steerable sheath such that a distal end of the steerable sheath is controllably deflected. The robotic catheter cradle may include an axial drive module operably coupled to the robotic catheter cradle to robotically translate the steerable sheath. The receiving module, the steering drive module, and the axial drive module may be configured such that the steerable sheath may, without the distal end being withdrawn from the patient, be removed by a human operator from the receiving module into a manual mode that permits operation mechanically independent of the robotic catheter cradle.

The at least one interface may be configured to be manually manipulated by the human operator. Manual introduction of the steerable sheath into the patient may include insertion of the distal end into the patient independent of an introducer associated with the robotic catheter cradle.

The apparatus may further include a control module configured to control motion of the steerable sheath relative to a proximal end of a dilator coupled to the patient.

Translate the steerable sheath may include translating the receiving module in a direction substantially parallel to the longitudinal axis.

In an illustrative aspect, an apparatus may include a receiving module extending along a longitudinal axis and configured to releasably receive at least a handle of a steerable sheath after a distal end of the steerable sheath has been manually introduced into a patient independently of the receiving module. The apparatus may include a steering drive module configured to robotically operate at least one interface of the handle to selectively actuate at least one guidewire of the steerable sheath such that a distal end of the steerable sheath is controllably deflected. The apparatus may include an axial drive module configured to robotically translate the steerable sheath.

The receiving module, the steering drive module, and the axial drive module may be configured such that the steerable sheath may, without the distal end being withdrawn from the patient, be removed by a human operator from the receiving module into a manual mode that permits mechanically independent manual operation of the steerable sheath from the handle to the patient.

The at least one interface may be configured to be manually manipulated by a human operator. The at least one interface may include a collar rotatable about the handle. The at least one interface may include a linear slide. The at least one interface may include a lever. The at least one interface may include an electronic touch input.

Manual introduction of the steerable sheath into the patient may include insertion of the distal end into the patient independent of an introducer associated with at least one of the receiving module, the steering drive module, and the axial drive module.

The axial drive module may be configured to robotically retract the steerable sheath.

The control module may be configured to control motion of the steerable sheath relative to a dilator coupled to the patient. The control module may be configured to control motion of the steerable sheath relative to a proximal end of the dilator. The dilator may be sutured to the patient.

The motion may include at least one of insertion and extraction. The motion may include rotation about an axis of rotation parallel to the longitudinal axis.

The receiving module may include a cradle configured to releasably receive the handle. The receiving module may include a frame rotatably suspending the cradle such that the cradle rotates about the axis of rotation relative to the frame.

Translate the steerable sheath may include translating the receiving module in a direction substantially parallel to the longitudinal axis.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

1-5. (canceled)

6. An apparatus comprising:
a receiving module extending along a longitudinal axis and configured to releasably receive at least a handle of a steerable sheath after a distal end of the steerable sheath has been manually introduced into a patient independently of the receiving module;
a steering drive module configured to robotically operate at least one interface of the handle to selectively control an electrically powered actuator module of the handle to actuate at least one guidewire of the steerable sheath such that a distal end of the steerable sheath is controllably deflected; and,
an axial drive module configured to robotically translate the steerable sheath.

7. The apparatus of claim 6, wherein the receiving module, the steering drive module, and the axial drive module are configured such that the steerable sheath may, without the distal end being withdrawn from the patient, be removed by a human operator from the receiving module into a manual mode that permits mechanically independent manual operation of the steerable sheath from the handle to the patient via the at least one interface.

8. The apparatus of claim 6, wherein the at least one interface is configured to be manually manipulated by a human operator.

9. The apparatus of claim 8, wherein the at least one interface comprises a collar rotatable about the handle.

10. The apparatus of claim 8, wherein the at least one interface comprises a linear slide.

11. The apparatus of claim 8, wherein the at least one interface comprises a lever.

12. The apparatus of claim 8, wherein the at least one interface comprises an electronic touch input.

13. The apparatus of claim 6, wherein manual introduction of the steerable sheath into the patient comprises insertion of the distal end into the patient independent of an introducer associated with at least one of the receiving module, the steering drive module, and the axial drive module.

14. The apparatus of claim 6, wherein the axial drive module is configured to robotically retract the steerable sheath.

15. The apparatus of claim 6, further comprising a control module configured to control motion of the steerable sheath relative to a dilator coupled to the patient.

16. The apparatus of claim 15, wherein the control module is further configured to control motion of the steerable sheath relative to a proximal end of the dilator, and the dilator is sutured to the patient.

17. The apparatus of claim 15, wherein the motion comprises at least one of insertion and extraction.

18. The apparatus of claim 15, wherein the motion comprises rotation about an axis of rotation parallel to the longitudinal axis.

19. The apparatus of claim 18, wherein the receiving module comprises:
a cradle configured to releasably receive the handle; and,
a frame rotatably suspending the cradle such that the cradle rotates about the axis of rotation relative to the frame.

20. The apparatus of claim 6, wherein translate the steerable sheath comprises translating the receiving module in a direction substantially parallel to the longitudinal axis.

21. The apparatus of claim 6, wherein the receiving module further comprises a monitor configured to monitor a visual display of a status in the steerable sheath.

22. The apparatus of claim 21, wherein the monitor comprises an optical sensor.

23. The apparatus of claim 21, wherein the monitor comprises a magnetic sensor.

24. The apparatus of claim 6, wherein the steerable sheath further comprises a battery operably coupled to supply power to the actuator of the handle.

25. The apparatus of claim 6, wherein the steerable sheath is sutured into a blood vessel of the patient.
